(19) United States
(12) Patent Application Publication
(10) Pub. No.: US 2014/0094512 A1
(43) Pub. Date: Apr. 3, 2014

(54) METHOD OF MODULATING THE DEGREE OF ADIPOSE TISSUE DEPOSITED INTRAMUSCULARLY

(71) Applicants: Nikolas Gunkel, Heidelberg (DE); Thorsten Meyer, Wiesbaden (DE); John Michael Graettinger, Baldwin City, KS (US)

(72) Inventors: Nikolas Gunkel, Heidelberg (DE); Thorsten Meyer, Wiesbaden (DE); John Michael Graettinger, Baldwin City, KS (US)

(21) Appl. No.: 14/037,966

(22) Filed: Sep. 26, 2013

(30) Foreign Application Priority Data
Oct. 2, 2012 (EP) 12186985.3

(51) Int. Cl. A23K 1/16; A23K 1/18; C07D 311/04

(52) U.S. Cl. CPC A23K 1/1618; C07D 311/04; A23K 1/1813
USPC 514/456; 549/405

(57) ABSTRACT

The present invention relates to compositions and methods for modulating the degree of adipose tissue deposited intramuscularly in cattle by administration of a retinoic receptor antagonist or inverse agonist and compounds for use in such method.

### FIELD OF THE INVENTION

The present invention relates to methods for increasing marbling in cattle.

### BACKGROUND OF THE INVENTION

Marbling is the common name used to describe the adipose tissue (i.e. fat) that is embedded in the connective tissue between the fascicules of muscles of cattle. Deposition of fat within muscles is a function of both genetics and nutrition. Veal has little to no marbling since young cattle develop subcutaneous fat first, kidney, pelvic and heart (KPH) fat second, inter-muscular (between the muscle, or “seam fat”) third and then intramuscular fat—“marbling”—last during the finishing phase of feedlot cattle immediately prior to slaughter.

Independent of its physiological function, the accretion of intramuscular fat is desirable in cattle because it is positively associated with the juiciness and flavor and tenderness of meat. Marbling is considered to be an indicator of palatability, with higher scores indicating better eating quality and result in higher quality grades—for beef—(USDA Prime and Choice) than lower marbling scores (USDA Select and Standard). Marbling scores in beef are determined by the amount and distribution of flecks of fat within the lean of the rib eye. The marbling scores, from lowest to highest, are: practically devoid, traces, slight, small, modest, moderate, slightly abundant, moderately abundant, and abundant. Marbling scores within each classification are further subdivided into degrees ranging from 0 to 100, which is necessary for determining the final quality grade.

Although beef cattle are currently fed high energy diets for extended periods of time in an attempt to assure a desirable degree of marbling, insufficient development of marbling and the excess deposition of subcutaneous fat (i.e. fat found under the skin) commonly referred to as “back-fat” are among the top challenges of the cattle industry.

Many proposals to improve marbling have been presented so far.

For example, the Japanese patent application JP 6022704 describes a method to improve the quality grade of beef, its fat marbling, firmness, flavor, tenderness, etc. by supplying feed containing calcium salt of fatty acid having a specific composition to cattle.

A short duration withdrawal followed by full feeding has been reported to result in an increased marbling score (Mir P. S. et al., Livestock Science 116 (2008):22-29).

U.S. Pat. No. 7,452,559, discloses a method of improving the fat marbling standard of meat by daily oral administration of oil-coated vitamin C crystalline powder to cattle.

The supplementation of cattle diet with ursodeoxycholic acid has been reported to lead to increased marbling (Irie et al., J. Anim Sci. 2011 89:4221-4226).

Dietary conjugated linoleic acid has been reported to increase marbling in pork (Barnes et al., J. Anim Sci. 2012 90:1142-1149).

Despite these suggested methods none of these has resulted in a product for increasing marbling.

Feeding diets that are low in vitamin A or vitamin A restriction for long periods in beef and dairy steers increased intramuscular or marbling adipose deposition without influencing other adipose depots (Gorocica-Buenfil et al., J. Anim Sci. 2007 85:2230-2242, Kruk et al. Livestock Science 2008 19:12-21,). However, a recent study of long-term vitamin A restriction in beef cattle failed to show an influence on marbling deposition (Gorocica-Buenfil et al., J. Anim Sci. 2008. 86:1609-1616). Vitamin A is required for normal visual function, maintenance of healthy epithelial tissues and mucous membranes, normal bone development and functional immunity in animals. Therefore a vitamin A restriction imposes a risk for the health and performance of animals.

Hence there remains a long-felt need for agents that will enhance marbling in cattle.

### SUMMARY OF THE INVENTION

The present invention provides a method for increasing marbling in cattle animals, comprising administering to a cattle animal an effective amount of a retinoic acid receptor inhibitor antagonist or inverse agonist.

In one embodiment, the invention provides such methods using a pan RAR, antagonist or inverse agonist.

In another embodiment, the invention additionally provides such methods using a selective RAR antagonist or inverse agonist.

In another embodiment the invention provides methods using a RAR antagonist or inverse agonist that is selective for RARα.

In one embodiment, the invention provides such method wherein the retinoic acid receptor antagonist or inverse agonist is administered orally to cattle.

In one embodiment, the invention provides such method wherein the retinoic acid receptor antagonist or inverse agonist is a retinoic acid receptor antagonist.

In one embodiment, the invention provides such method wherein the retinoic acid receptor antagonist is selected from a group of a pan retinoic acid receptor antagonist and a selective retinoic acid receptor a (alpha) antagonist.

In one embodiment, the invention provides such method wherein the retinoic acid receptor antagonist or inverse agonist is represented by the following structural Formula (I):

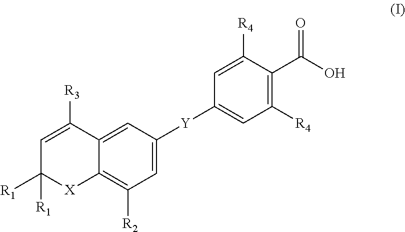

wherein
X is O, S or [C(Me)₂] and;
Y is —C≡C— or —CO—NH— and;
R₁ is hydrogen or C₁-C₄ alkyl and;
R₂ is hydrogen or halogen and;
R₃ is a phenyl optionally substituted with one or more R₄ or a heteroaryl where the heteroaryl has at least one nitrogen, oxygen or sulfur atom and is optionally substituted with one or more R₄ and;
R₄ is independently hydrogen, C₁-C₄ alkyl or halogen;
or a pharmaceutically acceptable salt thereof.

In one embodiment, the retinoic acid receptor antagonist or inverse agonist is represented by Formula (I) wherein

X is O or [C(Me)₂] and;
Y is —C≡C— or —CO—NH— and;
R₁ is hydrogen or C₁-C₄ alkyl and;
R₂ is hydrogen or halogen and;
R₃ is a phenyl optionally substituted with one or more R₄ or a heteroaryl where the heteroaryl has at least one nitrogen atom and is optionally substituted with one or more R₄ and;

R₄ is independently hydrogen, C₁-C₄ alkyl or halogen;

or a pharmaceutically acceptable salt thereof.

In one embodiment, the retinoic acid receptor antagonist or inverse agonist is represented by Formula (I) wherein

X is O or [C(Me)₂] and;
Y is —C≡C— or —CO—NH— and;
R₁ is hydrogen or methyl and;
R₂ is hydrogen or bromine and;
R₃ is p-tolyl or quinoline-3-yl and;
R₄ is independently hydrogen or fluorine;
or a pharmaceutically acceptable salt thereof.

In one embodiment the retinoic acid receptor antagonist or inverse agonist is represented by Formula (I) wherein X is O, R₁ is methyl, R₂ is bromine, R₃ is p-tolyl, R₄ is hydrogen and Y is —CO—NH—, or a pharmaceutically acceptable salt thereof.

In a separate embodiment the invention is directed to a compound of Formula (II):

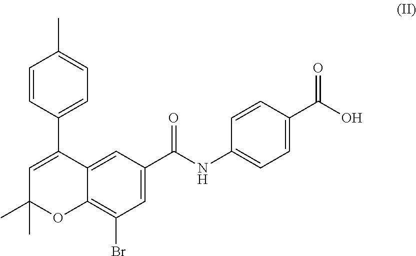 [SMILES: Cc1ccc(C2=CC(C)(C)Oc3c(Br)cc(C(=O)Nc4ccc(C(=O)O)cc4)cc32)cc1]

or a pharmaceutically acceptable salt thereof.

In another embodiment the invention is directed to a method for increasing marbling in cattle, comprising administering to cattle an effective amount of a compound of Formula (II).

This invention also is directed, in part, to a pharmaceutical composition. The pharmaceutical composition comprises a) a compound of Formula (II), and b) at least one excipient, and optionally, at least one agent which differ in structure from the component a).

This invention also is directed, in part, to cattle feed. The feed comprise a) at least one compound for use in this invention, and b) at least one regular feed base component, and optionally, at least one agent which differ in structure from the component a). In one embodiment the feed for cattle comprises a compound of Formula (II).

The RAR antagonist or inverse agonist, including the compounds of the Formula (I) and (II) and pharmaceutically acceptable salts thereof are hereinafter together referred to as “compound(s) for use in this invention”.

The method of increasing marbling in cattle animals by administration of the RAR antagonist or inverse agonist, including the compounds of the Formula (I) or (II) and pharmaceutically acceptable salts thereof to the animal is hereinafter referred to as “use or method according to the invention”.

Further benefits of Applicants' invention will be apparent to one skilled in the art from reading this specification.

### DESCRIPTION OF FIGURES

FIG. 1 shows the degree of Oil Red O staining of lipid droplets of 3T3-L1-preadipocyte as indicator of the degree of adipogenesis after administration of test compounds.

FIG. 2 shows the degree of Oil Red O staining of lipid droplets of bovine SVF cells preadipocyte as indicator of the degree of adipogenesis after administration of test compounds.

### DETAILED DESCRIPTION

The present invention provides methods to preferentially enhance deposition of intramuscular adipose tissue (marbling) by administering to a cattle animal a compound that pharmacologically acts as a retinoic acid receptor (RAR) antagonist or inverse agonist.

Retinoic acid is a metabolite of vitamin A (retinol) that mediates the functions of vitamin A required for growth and development.

Retinoic acid acts by binding to the retinoic acid receptor (RAR), which is bound to DNA as a heterodimer with the retinoid X receptor (RXR) in regions called retinoic acid response elements (RAREs). Binding of the retinoic acid ligand to RAR alters the conformation of the RAR, which affects the binding of other proteins that either induce or repress transcription of a nearby gene.

Retinoic acid receptors and retinoid X receptors are ligand-dependent transcription factors which regulate gene transcription by both up-regulating gene expression through binding RA-responsive elements and down-regulating gene expression by antagonizing the enhancer action of other transcription factors such as API.

Distinct RXRα, RXRβ and RXRγ isotypes and RARα, RARβ and RARγ isotypes are encoded by separate genes. Both RXR and RAR isotypes can be further expressed as several isoforms. The RARs function in vivo as RAR-RXR heterodimers. All-trans-Retinoic acid is the physiological hormone for the RARs.

Compounds that inhibit RAR expression, activity or signalling can be used as an RAR antagonist or inverse agonist.

An RAR antagonist or inverse agonist is a dominant negative molecule that prevents RAR activation, such as antibodies, proteins, small molecules and oligonucleotides that prevent or diminish ligand binding to RAR; ribozymes, antisense nucleic acid molecules, and nucleic acid molecules encoding negative regulatory transcription factors that prevent or reduce RAR expression, as well as cells or viruses containing such ribozymes and nucleic acid molecules, and selective antagonist or inverse agonists of RAR intracellular signalling.

As used herein, a “retinoic acid receptor antagonist” or “RAR antagonist” refers to a chemical compound and/or complexes of compounds having binding activity for the retinoic acid binding site of RAR. An RAR antagonist blocks the binding of retinoic acid to the receptor and thereby prevents or inhibits RAR activation.

As used herein, an “inverse agonist” is a chemical compound and/or complexes of compounds which is able to suppress the basal level of a retinoid receptor, for example an RAR, activity. Such activity can include homo-or heterodimerization and transacting transcriptional control of various genes whose regulation is normally responsive to RAR modulation.

As readily recognized by those of skill in the art, a variety of RAR antagonists and inverse agonists, both synthetic and naturally occurring, can be used in accordance with the present invention.

Preferably, the RAR antagonist and inverse agonist will be an RAR antagonist.

Some examples of structures and methods of making and using preferred RAR antagonists are provided in U.S. Pat. No. 5,776,699, which is incorporated by reference in its entirety.

In one embodiment the RAR antagonist is a RAR pan antagonist. In another embodiment the RAR antagonist is selective for RARα (alpha).

As used herein, the term “pan,” when used in reference to an RAR antagonist, refers to an RAR compound that binds to all forms of RAR, for example, RARα, RARβ and RARγ. A pan RAR antagonist, can exhibit differential binding between the forms of RAR so long as the pan RAR antagonist, binds to each RAR form such as RARα, RARβ and RARγ to a similar extent.

As used herein, the term “selective,” when used in reference to an RAR antagonist, refers to a compound that exhibits differential antagonist activity between one form of RAR over at least one other form of RAR. It is understood that any single RAR form or combination of RAR forms can be applicable to a selective RAR antagonist, or inverse agonist, so long as the compound does not significantly inhibit activity of or bind to all of the RAR forms. For example, a selective RAR antagonist dominantly or preferably binds to RARα and RARβ but not RARγ. Such an RAR antagonist is selective for RARα and RARβ. Similarly, an RAR antagonist selective for RARα binds to this form but not (or to a significant lower extent) to RARβ and RARγ.

The selectivity of an RAR antagonist or inverse agonist for RARα, RARβ and RARγ can be determined by in vitro tests well known in this art e.g. by an Gal4 reporter gene assay system as described e.g. in Bioorganic & Medicinal Chemistry 17 (2009) 4345-4359.

A selective RAR antagonist, or inverse agonist has at least about 10-fold higher affinity and/or antagonist or inverse agonist activity for one RAR form compared to at least one other RAR form. For example, a selective RAR antagonist can have at least about 15-fold higher, about 20-fold higher, about 25-fold higher, about 30-fold higher, about 35-fold higher, about 40-fold higher, about 45-fold higher, about 50-fold higher, about 60-fold higher, about 70-fold higher, about 80-fold higher, or about 90-fold higher binding activity or even higher for one RAR form compared to another.

A presently preferred class of RAR antagonists for use according to the invention are represented by the following structural Formula (I):

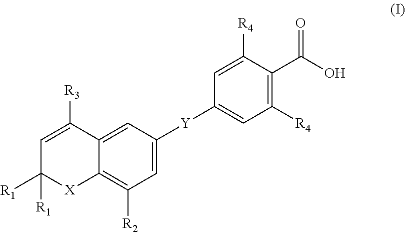

wherein
X is O, S or [C(Me)₂] and;
Y is —C≡C— or —CO—NH— and;
R₁ is hydrogen or C₁-C₄ alkyl and;
R₂ is hydrogen or halogen and;
R₃ is a phenyl optionally substituted with one or more R₄ or a heteroaryl where the heteroaryl has at least one nitrogen, oxygen or sulfur atom and is optionally substituted with one or more R₄ and;
R₄ is independently hydrogen, C₁-C₄ alkyl or halogen;
or a pharmaceutically acceptable salt thereof.

In one embodiment, the retinoic acid receptor antagonist or inverse agonist is represented by Formula (I) wherein

X is O or [C(Me)2] and;
Y is —C≡C— or —CO—NH— and;
R1 is hydrogen or C1-C4 alkyl and;
R2 is hydrogen or halogen and;
R3 is a phenyl optionally substituted with one or more R4 or a heteroaryl where the heteroaryl has at least one nitrogen atom and is optionally substituted with one or more R4 and;
R4 is independently hydrogen, C1-C4 alkyl or halogen;
or a pharmaceutically acceptable salt thereof.

In one embodiment, the retinoic acid receptor antagonist or inverse agonist is represented by Formula (I) wherein

X is O or [C(Me)2] and;
Y is —C≡C— or —CO—NH— and;
R1 is hydrogen or methyl and;
R2 is hydrogen or bromine and;
R3 is p-tolyl or quinoline-3-yl and;
R4 is independently hydrogen or fluorine;
or a pharmaceutically acceptable salt thereof.

In one embodiment the retinoic acid receptor antagonist or inverse agonist is represented by Formula (I) wherein X is O, R1 is methyl, R2 is bromine, R3 is p-tolyl, R4 is hydrogen and Y is —CO—NH—, or a pharmaceutically acceptable salt thereof.

Non-limiting examples of RAR antagonists include:

the RARα-selective antagonist AGN 194574; (CAS 192817-14-4), J. Med. Chem 1997, 40, 2445
the RARα-selective antagonist BMS 195614; (CAS 182135-66-6), U.S. Pat. No. 5,559,248;
the RAR pan-antagonist AGN 193109; (CAS 171746-21-7), J. Med. Chem 1995) 38271(21):4764
the RAR α/β antagonist LE540; (CAS 1888645-44-5) J Biol Chem 1999, 274(22):15360-6 and
the RARα-selective antagonist 4-[[8-bromo-2,2-dimethyl-4-(p-tolyl)chromene-6-carbonyl]amino]benzoic acid.

In a preferred embodiment the RAR antagonist has the following structure:

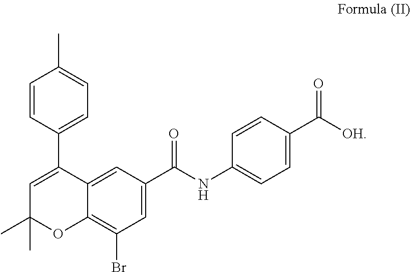 [SMILES: Cc1ccc(C2=CC(C)(C)Oc3c(Br)cc(C(=O)Nc4ccc(C(=O)O)cc4)cc32)cc1]

The compound of Formula (II) is a selective RAR alpha antagonist with an IC₅₀ in the low nM range (RARα-4 nM, RARβ-2979 nM and RARγ-2355 nM) as determined in a luciferase based RA responsive reporter assay. The compound of Formula (II) lacks any significant agonistic activity on RARs.

In addition to the compounds referred to herein, other agents that are known to have RAR antagonist and/or inverse agonist activity are also anticipated to be useful as marbling enhancer in the invention of the present application.

Additional RAR antagonist agents, that can be used in the method according to the current invention can be identified and tested by methods known to those skilled in the art. Such methods of screening for RAR antagonists include, but are not limited to, receptor binding, receptor transactivation and transactivation competition assays.

Receptor binding can be determined by competition assays using recombinant RAR proteins according to known methods. Receptor transactivation can be determined by those skilled in the art, for example, by transfecting cells with a RAR receptor and a reporter gene and assaying the transfected cells for the reporter gene product. The antagonist activity of potential RAR antagonists can be evaluated by known methods, for example, by a transactivation competition assay. Such antagonists can be selected and screened at random, or can be rationally selected or rationally designed using protein modelling techniques.

In vitro 3T3-L1 preadipocyte cell differentiation assays, e.g. the commercially available Adipolysis Assay Kit, available from Cayman Chemical Company, Ann Arbor, Mich., can be used to investigate the adipogenic effect on 3T3L1 mouse adipocytes cell differentiation by quantification of Oil Red O enriched in lipid droplets of 3T3L1 adipocytes.

Agents can be rationally selected. As used herein, an agent is said to be “rationally selected” when the agent is chosen based on the physical structure of a known ligand of a retinoid receptor or a functional homodimeric or heterodimeric retinoid receptor. For example, assaying compounds possessing a retinol-like structure would be considered a rational selection since retinol-like compounds are known to bind to a variety of retinoid receptor heterodimers.

Since highly purified RXR and RAR proteins are now available, X-ray crystallography and NMR-imaging techniques can be used to identify the structure of the ligand binding site present on these proteins and, by extension, that which is specifically present on the retinoid receptors. Utilizing such information, computer modelling systems are now available that allows one to “rationally design” an RAR antagonist capable of binding to such a defined structure.

The use of computer modelling can also be used to screen for antagonists of the RAR using known X-ray crystal structures of ligand-bound receptors. Using data from crystal structures, the ideal binding mode of the RAR antagonists is determined and a correlation between the structure of the compound and its effect of biological activity derived. Several general approaches exist for determining the three-dimensional structure activity relationships of compounds and receptors. For example, CATALYST®, DISCO, COMFA, and Apex3D are non-limiting examples of such approaches. See generally WO 98/04913.

In another screening assay, transgenic animals, e.g., mice, and cell lines, that are altered in their expression of one or more of RAR and RXR receptors can be made as described previously (Krezel, W., et al., Proc. Natl. Acad. Sci. USA 93:9010-9014 (1996)) and can be used to identify antagonists of specific members of the RAR/RXR class of receptors using methods described previously (WO 94/26100). In such an assay, the agent which is to be tested will be incubated with one or more of the transgenic cell lines or mice or tissues derived therefrom. The level of binding of the agent is then determined, or the effect the agent has on biological systems or gene expression is monitored, by techniques that are routine to those of ordinary skill.

Other methods for determining the antagonistic activities of a candidate ligand which are routine in the art can also be used in carrying out the present invention. In performing such assays, one skilled in the art will be able to determine which RAR receptor subtype(s), an agent binds to, what specific receptor(s) are utilized by a given compound, and whether the agent is an agonist or antagonist of the given receptor(s).

An effective amount of the RAR antagonist or inverse agonist is administered to the cattle animal in the method according to the invention. An “effective amount” of a compound is generally considered to be that which invokes a response; preferably in the absence of excessive side effects. A response in this case is considered an increase in marbling degree of cattle compared to an untreated control group. In one embodiment the marbling degree of carcass from cattle is increased by at least 10, 20, 30 40 or 50 degrees.

The RAR antagonist or inverse agonist can be administered via various administration routes and in specific dosage forms that are especially suitable for such administration routes. Typically, the RAR antagonist or inverse agonist is administered orally. In some embodiments, the RAR antagonist or inverse agonist is added to the intended recipient animal's drinking water. In other embodiments, the RAR antagonist or inverse agonist is added to the intended recipient's feed.

Oral dosage forms suitable for oral administration comprise liquids (e.g. drench or drinking water formulations), semi-solids (e.g. pastes, gels), and solids (e.g. tablets, capsules, powders, granules, chewable treats, premixes and medicated blocks).

Solid oral formulations are either administered directly via the mouth to an animal (tablet, capsule, time-release bolus) or mixed with the feed or via medicated feed blocks.

The RAR antagonist or inverse agonist may, for example, be intimately dispersed in the animal recipient's regular feed, used as a top dressing, or in the form of solid pellets, paste or liquid that is added to the finished feed. When the RAR antagonist or inverse agonist is administered as a feed additive (mixed with the feed), it may be convenient to prepare a “premix” in which the RAR antagonist or inverse agonist is dispersed in a liquid or solid carrier. Such a carrier can be any inert carrier material such as starch, lactose, as known in the pharmaceutical art or alternatively regular feed base components such as grains e.g. corn cobs. This “premix” is, in turn, dispersed in the cattle animal's feed using, for example, a conventional mixer allowing a more homogeneous distribution of the RAR antagonist in the feed. In one embodiment such premix for cattle feed comprises a compound of formula (I) or (II).

To the extent the RAR antagonist or inverse agonist is incorporated into feed, the feed mixture will vary depending on, for example, the type (e.g., species and breed), age, weight, activity, and condition of the intended recipient. For cattle, various feeds are well known in the art, and often comprise of cereals; grains, and soybean; flours of animal origin, such as fish flour; numerous by-products feeds; amino acids; mineral salts; vitamins; antioxidants; etc. These are regular feed base components. “Feed” refers particularly to food (for nutrition) given to the cattle animals, rather than that which they forage for themselves. It includes hay, straw, silage, compressed and pelleted feeds, oils and mixed rations, and sprouted grains and legumes.

Compound feeds are feedstuffs that are blended from various raw materials and additives. These blends are formulated according to the specific requirements of the target animal. They are manufactured by feed compounders as meal type, pellets or crumbles. Compound feeds can be complete feeds that provide all the daily required nutrients, concentrates that provide a part of the ration (protein, energy) or supplements that only provide additional micronutrients, such as minerals and vitamins.

In one embodiment the current invention is directed to cattle feed comprising an RAR antagonist or inverse agonist. Such cattle feed can be prepared by preparing a premix, as described above and then mixing such premix with regular feed base components. Alternatively the RAR antagonist or inverse agonist can be directly mixed with regular cattle feed base components. Preferably such cattle feed is compound feed as described above and is an energy rich diet as conventionally fed to cattle animals in feedlots during the finishing period. The cattle feed can be either grist, pellet or crumble type of feed. In one embodiment such cattle feed comprises a compound of formula (I) or (II).

In general, the RAR antagonist or inverse agonist can be incorporated into any feed that is available and used for the intended recipient cattle animal but especially in compound feeds. Alternatively the RAR antagonist or inverse agonist can be incorporated in an intra-ruminal (time-release) bolus. It is a veterinary delayed release delivery system which remains in the rumeno-reticular sac of a ruminant animal over an extended period of time and in which the therapeutically active substance has a predictable and delayed release pattern. Such intra-ruminal boluses are usually administered using a balling gun or another suitable device.

It is contemplated that the RAR antagonist or inverse agonist may alternatively be administered via non-oral dosage routes, such as topically (e.g., via a spot-on, pour-on or transdermal patch) or parenterally (e.g. as liquid subcutaneous injection, intravenous injection, intramuscular injection, etc. or as solid implants).

Implantable compositions are often administered as solid compressed pellets which are injected by an implanter equipped with a hypodermic needle. In cattle, implants are normally inserted in the ear or in other areas of the animal that are not for consumption and are discarded. The implanter needle is used to make a small self-sealing implant-receiving puncture beneath the skin at a suitable location on the body of the animal. Small pellets of a composition are forced through the needle and left under the skin as the needle is removed. This invention also is directed to a pharmaceutical composition comprising a compound of Formula (II). The compositions also may (and preferably will) comprise one or more pharmaceutically acceptable excipients.

Pharmaceutical compositions of the present invention may be manufactured by processes known in the art. These processes include, for example, a variety of known mixing, dissolving, granulating, and emulsifying, encapsulating, entrapping and lyophilizing processes.

Optimal formulation depends on, for example, the dosage route (e.g. oral, injection, topical). Solid dosage forms, for example, may be prepared by, for example, intimately and uniformly mixing the compounds with excipients (non-active inert ingredients) such as fillers, binders, lubricants, glidants, disintegrants, flavoring agents (e.g. sweeteners), buffers, preservatives, pharmaceutical-grade dyes or pigments and controlled release agents.

Further aspects regarding formulation of drugs and various excipients are found in, for example, Gennaro, A. R., et al., eds., Remington: The Science and Practice of Pharmacy (Lippincott Williams & Wilkins, 20th Ed., 2000).

The RAR antagonist or inverse agonist and the method according to the invention may be used in various cattle animals, which are raised to produce meat for human food (e.g. of genus Bos taurus or Bos indicus: e.g. cattle, buffalo, zebu, yak). Cattle are the most common type of large domesticated ungulates. They are a prominent modern member of the subfamily Bovinae, are the most widespread species of the genus Bos, and are most commonly classified collectively as Bos primigenius.

When the RAR antagonist or inverse agonist is orally administered, preferably a daily dosage form is used. The preferred total daily dose is typically greater than about 0.001 mg/kg (i.e., milligram of compound per kilogram body weight). In some embodiments, the daily dose is from about 0.01 to about 2 mg/kg. The RAR antagonist or inverse agonist can be administered to the cattle animal at a single time (e.g. delayed release device etc.). In general, however, the RAR antagonist or inverse agonist is administered over time, e.g. fed daily or continuously included in the animals feed for a certain period.

Although oral daily doses or continuous administration are typically preferred, it is contemplated that shorter or longer periods between doses can be used, depending on, for example, the cattle animal's metabolism of the RAR antagonist or inverse agonist. In one embodiment the RAR antagonist or inverse agonist is administered in intervals, e.g. every 2 days (“skip a day”), every 3 days, twice a week or weekly.

In some embodiments, for example, the RAR antagonist or inverse agonist is administered daily for at least 3 days, more typically daily for from about 10 to about 150 days, more typically daily for from about 14 to about 40 days, and still more typically daily for from about 20 to about 36 days.

In some particular embodiments, the RAR antagonist or inverse agonist is administered daily or in intervals for at least the first 30 days of the finishing period and/or for about the last 30 days prior to slaughter.

In some such embodiments, the RAR antagonist or inverse agonist is administered daily for from about the first 10 to about the first 60 days of the finishing period, and/or from about the last 10 to about 60 days of the finishing period prior to slaughter.

The term “finishing period” refers to the later stage of the growing period for an animal. During this period, cattle are typically confined in a feedlot. In some embodiments this period lasts for from about 50 to about 300 days, and depends on, for example, the starting body weight of the animal.

When administered via a subcutaneous implant, the preferred total daily dose of the RAR antagonist or inverse agonist is typically greater than about 0.001 mg/kg (i.e., milligram of compound per kilogram body weight).

If the RAR antagonist or inverse agonist is administered parenterally via an injection, the concentration in the dosage form preferably is sufficient to provide the desired therapeutically effective amount of the RAR antagonist or inverse agonist in a volume that is acceptable for parenteral administration.

Factors affecting the preferred dosage regimen may include, for example, the type (e.g., species and breed), age, size, sex, diet, activity, and condition of the intended recipient animal; the type of administration used (e.g., oral via feed, oral via drinking water, subcutaneous implant, other parenteral route, etc.); pharmacological considerations, such as the activity, efficacy, pharmacokinetic, and toxicology profiles of the particular RAR antagonist or inverse agonist administered; and whether the RAR antagonist or inverse agonist is being administered as part of a combination of active ingredients. Thus, the preferred amount of the compound can vary, and, therefore, can deviate from the typical dosages set forth above.

In some embodiments, the RAR antagonist or inverse agonist is administered in combination with other active ingredients. The administration of the other active ingredients typically can be before, simultaneous with, and/or after the administration of the RAR antagonist or inverse agonist. While the RAR antagonist or inverse agonist is typically administered over time, the other active(s) may be administered once, or, alternatively, over an amount of time, which may be the same as or different from the amount of time over which the RAR antagonist or inverse agonist is administered. To the extent that the administration is simultaneous, the combined actives may be part of the same dosage form and/or separate dosage forms.

### EXAMPLES

TABLE 1
RAR antagonist test compounds

| Compound No. | Structure | Reference |
|---|---|---|
| 1 | 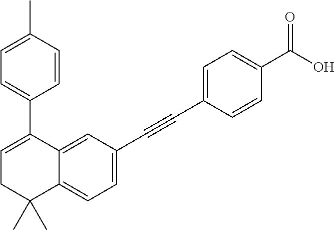 [SMILES: Cc1ccc(C2=CCC(C)(C)c3ccc(C#Cc4ccc(C(=O)O)cc4)cc32)cc1] | AGN 193109 J. Med Chem. 1995, 38, 4764 |
| 2 | 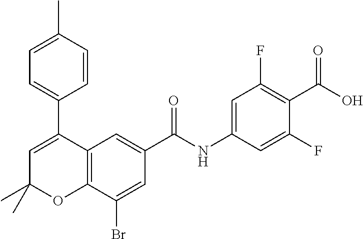 [SMILES: Cc1ccc(C2=CC(C)(C)Oc3c(Br)cc(C(=O)Nc4cc(F)c(C(=O)O)c(F)c4)cc32)cc1] | AGN 194574 J. Med Chem. 1997, 40, 2445 |
| 3 | 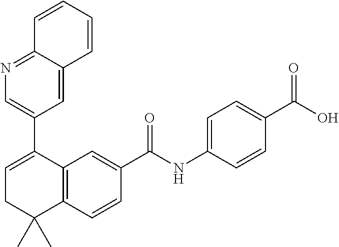 [SMILES: CC1(C)CC=C(c2cnc3ccccc3c2)c2cc(C(=O)Nc3ccc(C(=O)O)cc3)ccc21] | BMS 195614 U.S. Pat. No. 5,559,248 |
| 4 | 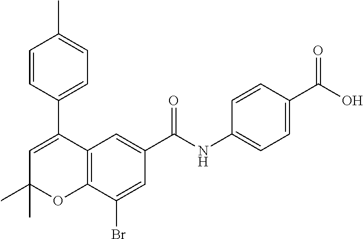 [SMILES: Cc1ccc(C2=CC(C)(C)Oc3c(Br)cc(C(=O)Nc4ccc(C(=O)O)cc4)cc32)cc1] | — |
| 5 | 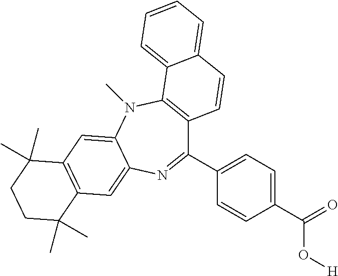 [SMILES: CN1c2cc3c(cc2N=C(c2ccc(C(=O)O)cc2)c2ccc4ccccc4c21)C(C)(C)CCC3(C)C] | LE540 Wako Pure Chemical Industries, Ltd. |

### Example 1

### Effect of RAR Antagonists on Retinoic Acid Induced Inhibition of 3T3-L1-Preadipocyte Differentiation

The effectiveness of the RAR alpha antagonists Compounds No. 2, 3 and 4 and the RAR pan antagonist Compound No. 1 in blocking the inhibitory effect of all-trans retinoic acid (ATRA) on 3T3-L1 preadipocyte differentiation was investigated.

In this experiment the “Oil Red O”-staining of lipid droplets is used as indicator of the degree of adipogenesis. After conveniently extraction of the dye from the lipid droplets the absorbance was measured with a microplate reader.

### Material and Methods

### a) Cell Culture and Differentiation:

3T3-L1-cells (passage 7) of one flask (75 cm²) were trypsinized by treating the adherent cells with 2 ml 0.25% Trypsin-EDTA. The reaction was stopped with fibroblast growth medium (DMEM/10% Newborn Calf Serum/1% Penicillin/Streptomycin) and 1 ml of this cellsuspension were added to each well of a 12-well-plate. Cells were incubated at 37° C./5% CO₂ in an incubator.

One day after cells reach confluency the fibroblast growth medium was removed and adipogenesis initiation medium (DMEM/10% FBS/1 μM Dexamethasone/0.5 mM IBMX/1 μg/ml Insulin) was added into 2 wells (i.e., in duplicate) without further substances (control), into 2 wells with 0.1 μM ATRA+ alternative 1 μM of compound No. 4, Compound No. 2, Compound No. 3 and 1 into 2 wells with 0.1 μM ATRA.

After incubation for two days, the adipogenesis initiation medium was removed and 1.5 ml adipogenesis medium (DMEM/10% FBS/1 μg/ml Insulin) containing the substances as indicated above was added per well. After another three days of incubation, the medium was replaced by adipogenesis medium without any additional substances. The culture medium was replaced every 2 or 3 days.

### b) Oil Red O Staining of Lipid Droplets

Nine days after addition of adipogenesis initiation medium, the medium was removed from all wells. Cells were fixed and stained with Oil Red O as follows:

Fix the cells in 10% formalin in PBS, remove formalin, dry completely, wash with PBS, dry at room temperature;
Prepare Oil red O working solution by diluting 3 parts Oil Red O stock solution with 2 parts dH2O;
Add Oil red O working solution for 1 hour;
Remove all Oil Red O and immediately add dH2O, wash with dH2O until water is colourless;
Wash with 60% isopropanol, dry completely at room temperature;
elute Oil red O by adding 60% isopropaniol/4% Triton X-100 solution for 1.5 hours;
transfer to 96 well plate;
measure absorption (optical density—OD) at 492 nm (Tecan Spectraflour plus)

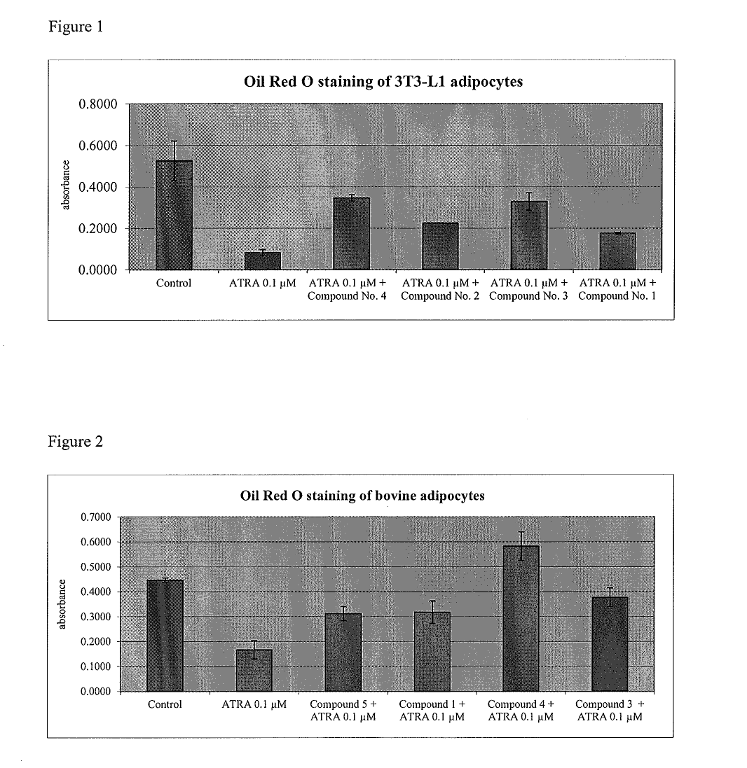

The absorbance is reported in Table 2 and FIG. 1 as the difference of the observed OD of extracted dye at 492 nm and a blank well OD value.

TABLE 2

| | | | | Std |
|---|---|---|---|---|
| | well1 | well2 | average | deviation |
| Control | 0.5932 | 0.4594 | 0.5263 | 0.0946 |
| ATRA 0.1 μM | 0.0925 | 0.0727 | 0.0826 | 0.0140 |
| ATRA 0.1 μM + compound | 0.3361 | 0.3571 | 0.3466 | 0.0148 |
| No. 4 1 μM | | | | |
| ATRA 0.1 μM + compound | 0.2264 | 0.2267 | 0.2266 | 0.0002 |
| No. 2 1 μM | | | | |
| ATRA 0.1 μM + compound | 0.3589 | 0.2996 | 0.3293 | 0.0419 |
| No. 3 1 μM | | | | |
| ATRA 0.1 μM + compound | 0.1736 | 0.1792 | 0.1764 | 0.0040 |
| No. 1 1 μM | | | | |

The amount of differentiated lipid-containing adipocytes is significantly reduced by the 0.1 μM ATRA.

All RAR antagonists used in this experiment diminished the anti-adipogenic effect of ATRA, although in neither case is the lipid content of control wells reached.

Determined by the amount of Oil Red O stainable lipid droplets, all tested compounds blocked the inhibitory effect of ATRA on 3T3-L1 preadipocyte differentiation.

### Effect of RAR Antagonists on Retinoic Acid Induced Inhibition of the Differentiation of Bovine SVF Cells to Adipocytes

The efficiency of the RAR antagonists compounds No. 1, 3, 4 and 5 in blocking the antiadipogenic effect of ATRA on bovine cells was investigated. The cells used in this experiment were from a primary culture of the stromal-vascular fraction isolated from intramuscular fat of an Angus steer.

To assess the adipocyte differentiation state, the cells of a 12-well plate were stained with “Oil Red O” and after conveniently extraction of the dye from the lipid droplets the absorbance was measured with a microplate reader.

### Material and Methods

### a) Cell Culture and Differentiation:

Cells of a bovine stromal-vascular fraction from intramuscular fat (passage 4) of one flask (75 cm²) were trypsinized by treating the adherent cells with 2 ml 0.25% Trypsin-EDTA. The reaction was stopped with growth medium (DMEM, 1% (v/v) of antibiotic solution, 50 μg/ml gentamicin sulfate, 33 μM biotin, 17 μM D-pantothenate, 100 μM (+)-sodium L-ascorbate and 10% FCS heat inactivated) and 1.5 ml of this cell suspension were added to each well of a 12-well-plate. Cells were incubated at 37° C./5% CO₂ in an incubator.

One day after cells reach confluency the growth medium was removed and adipogenesis initiation medium (growth medium/0.25 μM Dexamethasone/10 μg/ml Insulin/1 μM rosiglitazone) was added into 2 wells (i.e., in duplicate) without further compounds. Adipogenesis initiation medium additionally containing 0.1 μM ATRA was added into 2 wells without further compounds and alternative with 1 μM of compound No. 1, compound No. 3, compound No. 4 and compound No. 5 into 2 wells each of the 12-well plate.

After incubation for three days, the adipogenesis initiation medium was removed and 1.5 ml adipogenesis medium (growth medium/10 μg/ml Insulin/1 μM rosiglitazone) containing the substances as indicated above was added per well. The culture medium was replaced twice a week.

### b) Oil Red O Staining of Lipid Droplets

Thirteen days after addition of adipogenesis initiation medium the medium was removed and cells of the 12-well plate were fixed and stained with Oil Red O as previously described.

### Results

The absorbance is reported in Table 3 and FIG. 2 as the difference of the observed OD of extracted dye at 530 nm and a blank well OD value.

TABLE 3

| | | | | Std |
|---|---|---|---|---|
| | well 1 | well 2 | average | deviation |
| Control | 0.4407 | 0.4523 | 0.4465 | 0.0082 |
| ATRA 0.1 μM | 0.1409 | 0.1924 | 0.1667 | 0.0364 |
| Compound No. 5 1 μM + ATRA | 0.2915 | 0.3318 | 0.3117 | 0.0285 |
| 0.1 μM | | | | |
| Compound No 1 1 μM + ATRA | 0.2849 | 0.3493 | 0.3171 | 0.0455 |
| 0.1 μM | | | | |
| Compound No. 4 1 μM + ATRA | 0.5411 | 0.6229 | 0.5820 | 0.0578 |
| 0.1 μM | | | | |
| Compound No. 3 1 μM + ATRA | 0.3504 | 0.4044 | 0.3774 | 0.0382 |
| 0.1 μM | | | | |

The amount of Oil Red O stainable lipid droplets is clearly decreased in bovine cells cultured under the influence of 0.1 μM ATRA.

All testes RAR antagonists can significantly diminish the anti-adipogenic effect of ATRA, although the lipid accumulation of control cells is not reached in cells cultured with Compound No. 5, 1 and 3.

### Example 3

### In Vivo Study Cattle

### Enhancement of Marbling in Beef Cattle Steers

The objective of this study is to determine if feeding the test compound No. 4 of Table 1 (compound of Formula II) during the first 28 days of a 140 day finishing period will improve the marbling score in beef steers.

The study will consist of 2 treatments with 24 cattle animals per treatment housed individually. The treatments will consist of a control treatment in which animals will receive the base finishing diet throughout the study and the test compound treatment in which animals will receive the test compound for the first 28 days of the study in addition to the base finishing diet. After the first 28 days on trial all animals will receive the base finishing diet. Cattle of similar breed, genetic makeup and uniform body weight (350 to 375 kg) will be used.

Prior to study initiation initial measurements of bodyweight (BW), ultrasound fat thickness (UFT), and ultrasound marbling score (UMS) of each steer will be obtained. An equation will be used to predict the final marbling score of each test animal at slaughter.

Cattle will be ranked by predicted marbling score (low to high) and randomized to treatment groups. Cattle will be housed individually in 48 pens with dirt surfaces. Water will be available in each pen at all times.

Breeds of cattle will be typical of the beef industry and those commonly used to produce carcasses for the boxed beef industry.

Ages of cattle used in these studies will be typical of industry practices and may range between approximately 12 and 26 months of age.

All diet ingredients of the base finishing diet used in the study are representative of feeds used in finishing rations for cattle. Diets will be fed once a day and recorded daily. Weigh-backs will be conducted if feed is off condition. All feed and water will be offered ad libitum throughout the study.

The teat compound will be administered in a solution (30 mg/mL) at a dosage of 2 mg test compound/kg BW twice per week. Ultrasound data (fat thickness and marbling score) will be obtained prior to study initiation, on days 28, 56, 84, 112 and at the end of the study. If the test compound appears to have increased marbling by a very large amount by day 112, a decision will be made to additionally feed the test compound during the last 28 day period prior to slaughter.

Hot carcass weight will be recorded during harvest.

Grade information including their individual components will be collected on “called” USDA Quality and Yield Grades. Specific carcass measurements including marbling score, ribeye area, and backfat will also be recorded. Carcass data will be collected and recorded according to guidelines outlined in Recommended Procedures for Beef Carcass Evaluation and Carcass Contests, American Meat Science Association (AMSA), 3rd Edition, 1990.

### Example 4

### Synthesis of 4-[[8-bromo-2,2-dimethyl-4-(p-tolyl)chromene-6-carbonyl]amino]benzoic acid (Compound No. 4)

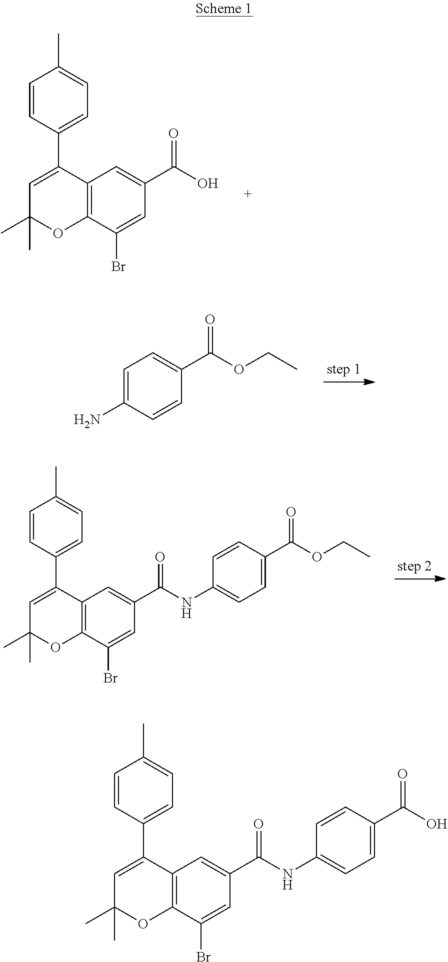

The starting material 8-bromo-2,2-dimethyl-4-(p-tolyl)chromene-6-carboxylic acid can be prepared following the procedure reported by Teng et al. in J. Med. Chem. 1997, 40 (16), 2445-2451.

### Step 1

To a stirred mixture of 8-bromo-2,2-dimethyl-4-(p-tolyl)chromene-6-carboxylic acid (56 mg, 0.15 mmol) in dichloromethane (4 mL) was added 4-(dimethylamino)-pyridine (44 mg, 0.36 mmol) and ethyl-4-aminobenzoate (37 mg, 0.225 mmol). 1-Ethyl-3-(3-dimethylaminopropyl)carbodiimide (43 mg, 0.225 mmol) was then added and the reaction mixture was stirred at room temperature for 24 hours. The resulting solution was directly submitted to column chromatography on silica gel using dichloromethane as the mobile phase. Ethyl 4-[[8-bromo-2,2-dimethyl-4-(p-tolyl)chromene-6-carbonyl]amino]benzoate (69 mg, 0.133 mmol) was obtained as a white solid. ¹H NMR (300 MHz, CDCl₃) □ 8.03 (2H, d, J=8.7 Hz), 7.91 (1H, d, J=2.1 Hz), 7.70 (1H, bs), 7.65 (2H, d, J=8.7 Hz), 7.50 (1H, d, 2.1 Hz), 7.25 (4H, s), 5.70 (1H, 1s), 4.359 (2H, q, J=7.1 Hz), 2.39 (3H, s), 1.56 (6H, s), 1.39 (3H, t, J=7.1 Hz).

### Step 2

To a solution of ethyl 4-[[8-bromo-2,2-dimethyl-4-(p-tolyl)chromene-6-carbonyl]amino]benzoate (130 mg, 0.25 mmol) in a 4:1 v/v mixture of tetrahydrofuran and ethanol (5 mL) was added a 4 molar aqueous solution of sodium hydroxide (1 mL) and the reaction mixture was placed in a microwave at 80° C. for 100 minutes. The reaction mixture was then allowed to attain room temperature and was acidified to pH4 with 1 N hydrochloric acid. All volatiles were removed in vaccuo and ethyl acetate (10 mL) and water (2 mL) were then added. The aqueous phase was separated and extracted with ethyl acetate (1×10 mL) and the combined organic phases washed with brine (5 mL) and dried over Na₂SO₄. Column chromatography on silica gel (dichloromethane/diethyl ether, 5:1 then 2:1, Ethyl acetate and then methanol/dichloromethane 1:9) gave 4-[[8-bromo-2,2-dimethyl-4-(p-tolyl)chromene-6-carbonyl]amino]benzoic acid as a solid (85 mg, 0.173 mmol). ¹H NMR (400 MHz, (CD₃)₂CO) □ 9.77 (1H, bs), 8.11 (1H, d, J=2.0 Hz), 7.99 (2H, d, J=8.7 Hz), 7.88 (2H, d, J=8.7 Hz), 7.70 (1H, d, J=2.0 Hz), 7.28 (4H, s), 5.88 (1H, s), 2.38 (3H, s), 1.57 (6H, s).

1. A method for increasing marbling in cattle, comprising administering to a cattle animal an effective amount of a retinoic acid receptor (RAR) antagonist or RAR inverse agonist.

2. The method according to claim 1 wherein the RAR antagonist is selected from a group of a pan RAR antagonist and a selective RAR alpha antagonist.

3. The method according to claim 1 wherein the RAR antagonist is represented by the following Formula (I): 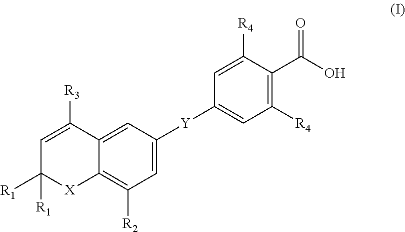
wherein
X is O, S or [C(Me)₂] and;
Y is —C≡C— or —CO—NH— and;
R₁ is hydrogen or C₁-C₄ alkyl and;
R₂ is hydrogen or halogen and;
R₃ is a phenyl optionally substituted with one or more R₄ or a heteroaryl where the heteroaryl has at least one nitrogen, oxygen or sulfur atom and is optionally substituted with one or more R₄ and;
R₄ is independently hydrogen, C₁-C₄ alkyl or halogen; or a pharmaceutically acceptable salt thereof.

4. The method according to claim 3 wherein
X is O or [C(Me)₂] and;
Y is —C≡C— or —CO—NH— and;
R₁ is hydrogen or C₁-C₄ alkyl and;
R₂ is hydrogen or halogen and;
R₃ is a phenyl optionally substituted with one or more R₄ or a heteroaryl where the heteroaryl has at least one nitrogen atom and is optionally substituted with one or more R₄ and;
R₄ is independently hydrogen, C₁-C₄ alkyl or halogen; or a pharmaceutically acceptable salt thereof.

5. The method according to claim 4 wherein
X is O or [C(Me)₂] and;
Y is —C≡C— or —CO—NH— and;
R₁ is hydrogen or methyl and;
R₂ is hydrogen or bromine and;
R₃ is p-tolyl or quinoline-3-yl and;
R₄ is independently hydrogen or fluorine; or a pharmaceutically acceptable salt thereof.

6. The method according to claim 4 wherein in the compound of Formula (I) X is O, R₁ is methyl, R₂ is bromine, R₃ is p-tolyl, R₄ is hydrogen and Y is —CO—NH.

7. The method according to claim 1 wherein the RAR antagonist or inverse agonist is administered orally to the cattle animal.

8. A compound of Formula (II) or a pharmaceutically acceptable salt thereof

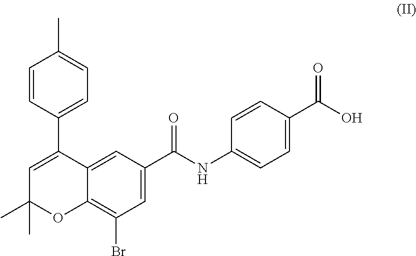 [SMILES: Cc1ccc(C2=CC(C)(C)Oc3c(Br)cc(C(=O)Nc4ccc(C(=O)O)cc4)cc32)cc1]

9. A pharmaceutical composition comprising a compound of Formula (II) and at least one excipient.

10. A premix for incorporation in feed comprising an RAR antagonist or inverse agonist and a liquid or solid carrier.

11. The premix for incorporation in feed comprising the RAR antagonist or inverse agonist of Formula (I): 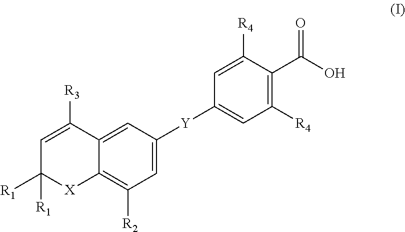
wherein
X is O or [C(Me)₂] and;
Y is —C≡C— or —CO—NH— and;
R₁ is hydrogen or methyl and;
R₂ is hydrogen or bromine and;
R₃ is p-tolyl or quinoline-3-yl and;
R₄ is independently hydrogen or fluorine; or a pharmaceutically acceptable salt thereof.

12. The premix according to claim 11 wherein the RAR antagonist or inverse agonist is the compound of Formula (II) or a pharmaceutically acceptable salt thereof

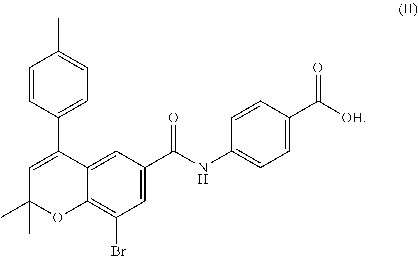 [SMILES: Cc1ccc(C2=CC(C)(C)Oc3c(Br)cc(C(=O)Nc4ccc(C(=O)O)cc4)cc32)cc1]

13. A feed for cattle comprising an RAR antagonist or inverse agonist and at least one regular feed base component.

14. A feed for cattle comprising a compound of claim 8 and at least one regular feed base component.
